(19) United States
(12) Patent Application Publication
(10) Pub. No.: US 2010/0278897 A1
(43) Pub. Date: Nov. 4, 2010

(54) INTRAOCULAR BIOACTIVE AGENT DELIVERY SYSTEM WITH MOLECULAR PARTITIONING SYSTEM

(75) Inventors: Ruiwen SHI, Irvine, CA (US); Patrick M. Hughes, Aliso Viejo, CA (US)

Correspondence Address: ALLERGAN, INC., 2525 DUPONT DRIVE, T2-7H, IRVINE, CA, 92612-1599, US

(73) Assignee: ALLERGAN, INC., Irvine, CA (US)

(21) Appl. No.: 12/434,541

(22) Filed: May 1, 2009

(51) Int. Cl. A61K 31/496; A61F 2/00; A61P 27/02

(52) U.S. Cl. 424/427; 514/253.11

(57) ABSTRACT

The present disclosure generally provides intraocular implants including at least one therapeutic bioactive agent and a molecular partitioning system. The molecular partitioning system comprises at least two phases wherein the first phase has an inherent viscosity equal or greater than the inherent viscosity of a second phase. The molecular partitioning system allows the intraocular implants to controllably release the at least one therapeutic bioactive agent into the surrounding tissues once implanted.

### FIELD OF THE INVENTION

The present invention relates to intraocular implants including a molecular partitioning system comprising a first phase having a first mean viscosity; a second phase having a second mean viscosity; and at least one bioactive agent.

### BACKGROUND

Historically, treatment of eye conditions has usually been effected through the use of applied, topical ophthalmic drugs in either liquid or ointment form. However, in many instances, it is preferable to release a pharmaceutical agent at a controlled and/or continuous rate over a prolonged period of time in order to obtain a desired pharmacological effect. It is well known that such continuous delivery of a drug, or an active agent, is not obtainable through the use of liquid or ointment application, despite periodic application of these medications. Even with the controlled dispensing of liquid eye drops, for example, the level of medication in the eye varies dramatically because of the washing effect of tears which can substantially decrease the amount of available medication until the next application of drops.

As such, delivery of drugs to different regions of the eye, such as the retina, vitreous and uveal tract is typically achieved by high systemic dosing, intra-ocular injections or other heroic measures. Penetration of systemically administered drugs into the retina is severely restricted by the blood-retinal barrier (BRB) for most compounds. Although intraocular injection, such as intravitreal injections, resolves some constraints posed by the BRB and significantly reduces the risk of systemic toxicity, intraocular injection techniques may result in retinal detachment, physical damage to the lens, exogenous endophthalmitis, and also may result in high pulsed concentrations of drug at the lens and other intraocular tissues.

Another complication is that compounds are eliminated from the vitreous by diffusion to the retro-zonular space with clearance via the aqueous humor or by trans-retinal elimination. Most compounds utilize the former pathway while lipophilic compounds and those with trans-retinal transport mechanisms will utilize the latter. Unfortunately, compounds that are eliminated across the retina have extremely short half-lives. Hence, for these compounds it is difficult to maintain therapeutic concentrations by direct intraocular injection, and therefore, frequent injection is often required.

Additionally, the rapid elimination of retinaly cleared compounds makes formulation of controlled delivery systems challenging. For example, tyrosine kinase inhibitors (TKIs) may possess extremely short intraocular half-lives, and thus, may pose a challenge to the formulation of controlled delivery systems. Small molecule TKIs given by intraocular administration, let alone, intraocular implants containing TKIs are very rare and quite difficult to formulate.

It would be advantageous to provide implantable drug delivery systems to an eye, such as intraocular implants, and methods of using such systems, that are capable of releasing a therapeutic agent at a sustained or controlled rate for extended periods of time and in amounts with few or no negative side effects.

The present description generally relates to intraocular implants and therapeutic use of such systems. In particular the present invention relates to an intraocular, tyrosine kinase inhibitor (TKI), controlled release drug delivery system for treatment of retinal diseases and conditions.

### SUMMARY

The present description generally provides ocular implants and implant systems, preferably intraocular implants, for the treatment of retinal diseases and conditions. The implants and implant systems include a molecular partitioning system comprising at least two different phases having different inherent, or mean viscosities and/or molecular weights and at least one therapeutic bioactive agent. The molecular partitioning system provides controlled release of the at least one therapeutic bioactive agent from the implants.

The implants and implant systems can release at least one therapeutic bioactive agent over a relatively long period of time, for example, for at least about one week or for example, between one week and one year, such as over two weeks, one month, two months or over three months or longer, after intraocular (i.e. intrascleral [such as subconjunctival] or intravitreal) administration of at least one therapeutic bioactive agent containing implant. Such extended release times facilitate successful treatment results. In addition, administering implants and implant systems to an intraocular location provides both a high, local therapeutic level of at least one therapeutic bioactive agent at the intraocular (retinal) target tissue and importantly eliminates or substantially eliminates presence of toxic bioactive agent intermediates and metabolites at the site of the intraocular target tissue.

In one example embodiment described herein are intraocular implants for treating an ocular condition, the implant comprising: a molecular partitioning system comprising a poly(D,L-lactide) phase having a first inherent viscosity; a poly(D,L-lactide-co-glycolide) phase having a second inherent viscosity; and at least one therapeutic bioactive agent; wherein the first mean viscosity is at least about four times greater than the second mean viscosity, and wherein the molecular partitioning system provides controlled release of the at least one therapeutic bioactive agent from the intraocular implant. In another example embodiment, the poly(D,L-lactide) phase has a first molecular weight and the poly(D,L-lactide-co-glycolide) phase has a second molecular weight wherein the first molecular weight is at least about four times greater than the second molecular weight.

In another example embodiment described herein are processes for making an intraocular implant having a molecular partition system comprising: dissolving a poly(D,L-lactide) polymer having a first mean viscosity; a poly(D,L-lactide-co-glycolide) polymer having a second mean viscosity; and at least one therapeutic bioactive agent in a solvent thereby forming a mixture; casting the mixture; evaporating the solvent thereby forming a polymeric film comprising the molecular partitioning system, the molecular partitioning system comprising a poly(D,L-lactide) phase having the first mean viscosity and a poly(D,L-lactide-co-glycolide) phase having the second mean viscosity; and extruding the polymer film thereby making the intraocular implant, wherein the first mean viscosity is at least about four times greater than the second mean viscosity and the molecular partitioning system provides controlled release of the at least one therapeutic bioactive agent from the intraocular implant. In still a further example embodiment, the extruding step is performed at a temperature of about 90° C.

Yet in a further example embodiment described herein are methods of treating an ocular condition comprising the steps of: (a) selecting a patient with an ocular condition in need of treatment; (b) providing an intraocular implant comprising a molecular partitioning system comprising a poly(D,L-lactide) phase having a first mean viscosity; a poly(D,L-lactide-co-glycolide) phase having a second mean viscosity; and at least one therapeutic bioactive agent, wherein the first mean viscosity is at least about four times greater than the second mean viscosity, and wherein the molecular partitioning system provides controlled release of the at least one therapeutic bioactive agent from the intraocular implant; (c) inserting the intraocular implant into a region of an eye; and (d) treating the ocular condition.

In yet another example embodiment, the poly(D,L-lactide) phase, the poly(D,L-lactide-co-glycolide) phase, and the at least one therapeutic bioactive agent are present at a ratio of about 60:20:20.

In still further example embodiments, the at least one therapeutic bioactive agent is a tyrosine kinase inhibitor having the structure

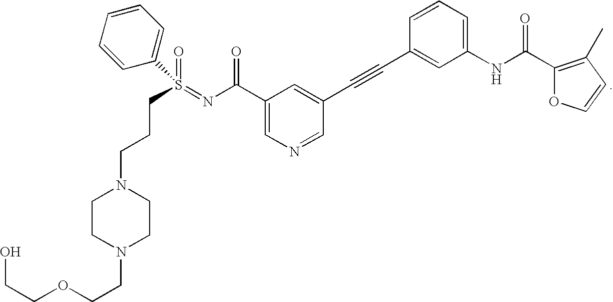

In other embodiments, the at least one therapeutic bioactive agent is greater than about 60% partitioned into said poly(D,L-lactide-co-glycolide) phase or is greater than about 75% partitioned into said poly(D,L-lactide-co-glycolide) phase. In other embodiments, the ocular implant is rod shaped.

Further described herein are processes of making an intraocular implant having a molecular partitioning system comprising: dissolving a poly(D,L-lactide) polymer having a mean viscosity between about 1.3 and about 1.7 dl/g; a poly(D,L-lactide-co-glycolide) polymer having a mean viscosity between about 0.32 and about 0.44 dl/g; and at least one bioactive agent in dichloromethane thereby forming a mixture; casting the mixture; evaporating the dichloromethane thereby forming a polymer film comprising the molecular partitioning system having a poly(D,L-lactide) phase and a poly(D,L-lactide-co-glycolide) phase; and extruding the polymeric film into rod shaped structures at a temperature of about 90° C. thereby making the intraocular implant, wherein the molecular partitioning system provides controlled release of the at least one bioactive agent from the intraocular implant. In one example embodiment, the poly(D,L-lactide) polymer, the poly(D,L-lactide-co-glycolide) polymer, and the at least one bioactive agent are present at a ratio of about 60:20:20.

### BRIEF DESCRIPTION OF THE DRAWINGS

Aspects of the present description are illustrated by the following drawings.

FIG. 1 graphically illustrates in vitro release profiles of Compound A from four different implant formulations. The release medium was 0.02% polysorbate 80 containing 10 mM phosphate buffered saline, pH7.4.

FIGS. 2A and 2B are example SEM images of the cross-sections of implants after 6 days of in vitro release. FIG. 2A is implant 1 and FIG. 2B is implant 5.

FIGS. 3A and 3B are example SEM images of the cross-sections of implants after 5 days in rabbit eyes. FIG. 3A is implant 1 and FIG. 3B is implant 5.

FIGS. 4A and 4B illustrate example shapes of the pores and the impact of the pores on the surface areas of the implants. FIG. 4A is implant 1 and FIG. 4B is implant 5.

FIG. 5 are GPC chromatograms of 20% Compound A loaded implants containing PLA and PLGA at a ratio of 50:50 before implantation and after being implanted in rabbit eyes for 5 days.

FIG. 6 are GPC chromatograms of 20% Compound A loaded implants containing PLA and PLGA at a ratio of 50:50 after 5 days in rabbit eyes and 6 days in the release medium at 37° C. in vitro.

### DEFINITION OF TERMS

Before proceeding it may be useful to define many of the terms used to describe embodiments according to the present description. Words and terms of art used herein should be first defined as provided for in this specification, and then as needed as one skilled in the art would ordinarily define the terms.

As used herein, “about” means plus or minus about ten percent of a number, parameter or characteristic described herein.

As used herein “biocompatible” shall mean any material that does not cause injury or death or induce an adverse reaction when placed in intimate contact with the implanted tissues. Adverse reactions include inflammation, infection, fibrotic tissue formation, cell death, or thrombosis.

As used herein, “biodegradable polymer” means a polymer or polymers which degrade in vivo, and wherein erosion of the polymer or polymers over time occurs concurrent with, or subsequent to, release of a drug or therapeutic agent. The terms “biodegradable” and “bioerodible” are equivalent and are used interchangeably herein. A biodegradable polymer may be a homopolymer, a copolymer, or a polymer comprising more than two different polymeric units. The polymer can be a gel or hydrogel type polymer, or mixtures or derivatives thereof.

As used herein “controlled release” refers to the release of at least one therapeutic bioactive agent, or drug, from an implant surface at a predetermined rate. Controlled release implies that the at least one therapeutic bioactive agent does not come off the implant surface sporadically in an unpredictable fashion and does not “burst” off of the implant upon contact with a biological environment (also referred to herein as first order kinetics) unless specifically intended to do so. However, the term “controlled release” as used herein does not preclude a “burst phenomenon” associated with deployment. In some example embodiments according to the present description an initial burst of at least one therapeutic bioactive agent may be desirable followed by a more gradual release thereafter. The release rate may be steady state (commonly referred to as “timed release” or zero order kinetics), that is the at least one therapeutic bioactive agent is released in even amounts over a predetermined time (with or without an initial burst phase) or may be a gradient release. A gradient release implies that the concentration of therapeutic bioactive agent released from the device surface changes over time.

As used herein, “molecular partitioning system” refers to the polymeric phase differentiation and sequestering that occurs in the implants described herein. The implants include at least a first polymer or co-polymer and a second polymer or co-polymer. The partitioning effect is believed to occur because the first polymer or co-polymer has a mean viscosity that is at least equal to or greater than the second polymer or co-polymer. For example, a difference in mean viscosity of greater than four can be useful. Other differences in mean viscosity between the first polymer or co-polymer and the second polymer or copolymer can be at least seven or at least ten. The difference in mean viscosity between the different polymers should allow the resulting polymer to remain stable once formed and provide the in vivo characteristics sought. This difference in mean viscosity causes the first and second polymer or co-polymer to partition into two different phases. The phases themselves are further defined when at least one therapeutic bioactive agent is added to the system. The at least one therapeutic bioactive agent has an affinity for one of the two phases and partitions itself into that phase more readily than the other. The resulting system has at least two phases one being polymer rich (having less of the therapeutic bioactive agent) and a bioactive agent rich phase (having more of the therapeutic bioactive agent). In the molecular partitioning systems described herein, the at least one therapeutic bioactive agent is greater than 60% partitioned into the drug rich phase. In another example embodiment, the at least one therapeutic bioactive agent is greater than 75% partitioned into the drug rich phase. In one example embodiment, the molecular partitioning system includes PLGA and PLA and the bioactive agent is partitioned into the PLGA phase.

As used herein, “ocular region” or “ocular site” means any area of the eyeball, including the anterior and posterior segment of the eye, and which generally includes, but is not limited to, any functional (e.g., for vision) or structural tissues found in the eyeball, or tissues or cellular layers that partly or completely line the interior or exterior of the eyeball. Specific examples of areas of the eyeball in an ocular region include the anterior chamber, the posterior chamber, the vitreous cavity, the choroid, the suprachoroidal space, the conjunctiva, the subconjunctival space, the episcleral space, the intracorneal space, the epicorneal space, the sclera, the pars plana, surgically-induced avascular regions, the macula, and the retina.

As used herein, “ocular condition” means a disease, ailment or condition which affects or involves the eye or one of the parts or regions of the eye. Broadly speaking the eye includes the eyeball and the tissues and fluids which constitute the eyeball, the periocular muscles (such as the oblique and rectus muscles) and the portion of the optic nerve which is within or adjacent to the eyeball.

For example, an anterior ocular condition is a disease, ailment or condition which affects or which involves an anterior (i.e. front of the eye) ocular region or site, such as a periocular muscle, an eye lid or an eye ball tissue or fluid which is located anterior to the posterior wall of the lens capsule or ciliary muscles. Thus, an anterior ocular condition primarily affects or involves the conjunctiva, the cornea, the anterior chamber, the iris, the posterior chamber (behind the retina but in front of the posterior wall of the lens capsule), the lens or the lens capsule and blood vessels and nerve which vascularize or innervate an anterior ocular region or site.

Thus, an anterior ocular condition can include a disease, ailment or condition, such as for example, aphakia; pseudophakia; astigmatism; blepharospasm; cataract; conjunctival diseases; conjunctivitis; corneal diseases;, corneal ulcer; dry eye syndromes; eyelid diseases; lacrimal apparatus diseases; lacrimal duct obstruction; myopia; presbyopia; pupil disorders; refractive disorders and strabismus. Glaucoma can also be considered to be an anterior ocular condition because a clinical goal of glaucoma treatment can be to reduce a hypertension of aqueous fluid in the anterior chamber of the eye (i.e. reduce intraocular pressure).

Alternatively, a posterior ocular condition is a disease, ailment or condition which primarily affects or involves a posterior ocular region or site such as choroid or sclera (in a position posterior to a plane through the posterior wall of the lens capsule), vitreous, vitreous chamber, retina, optic nerve (i.e. the optic disc), and blood vessels and nerves which vascularize or innervate a posterior ocular region or site.

Thus, a posterior ocular condition can include a disease, ailment or condition, such as for example, acute macular neuroretinopathy; Behcet's disease; choroidal neovascularization; diabetic uveitis; histoplasmosis; infections, such as fungal or viral-caused infections; macular degeneration, such as acute macular degeneration, non-exudative age related macular degeneration and exudative age related macular degeneration; edema, such as macular edema, cystoid macular edema and diabetic macular edema; multifocal choroiditis; ocular trauma which affects a posterior ocular site or location; ocular tumors; retinal disorders, such as central retinal vein occlusion, diabetic retinopathy (including proliferative diabetic retinopathy), proliferative vitreoretinopathy (PVR), retinal arterial occlusive disease, retinal detachment, uveitic retinal disease; sympathetic opthalmia; Vogt Koyanagi-Harada (VKH) syndrome; uveal diffusion; a posterior ocular condition caused by or influenced by an ocular laser treatment; posterior ocular conditions caused by or influenced by a photodynamic therapy, photocoagulation, radiation retinopathy, epiretinal membrane disorders, branch retinal vein occlusion, anterior ischemic optic neuropathy, non-retinopathy diabetic retinal dysfunction, retinitis pigmentosa, and glaucoma. Glaucoma can be considered a posterior ocular condition because the therapeutic goal is to prevent the loss of or reduce the occurrence of loss of vision due to damage to or loss of retinal cells or optic nerve cells (i.e. neuroprotection).

As used herein, “therapeutically effective amount” means level or amount of a therapeutic bioactive agent or drug needed to treat an ocular condition, or reduce or prevent ocular injury or damage without causing significant negative or adverse side effects to the eye or a region of the eye. In view of the above, a therapeutically effective amount of a bioactive agent, such as a Compound A, is an amount that is effective in reducing at least one symptom of an ocular condition.

As used herein “Compound A” refers to a tyrosine kinase inhibitor having a formula

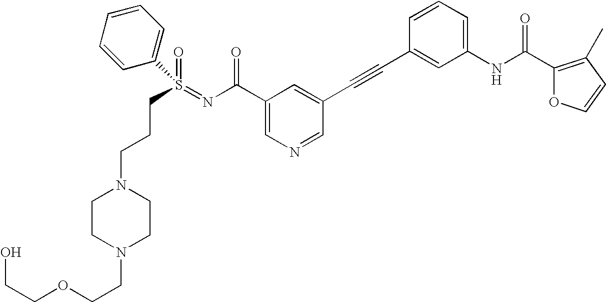

and all salts, prodrugs, esters, isomers, derivatives and analogues thereof.

### DETAILED DESCRIPTION

The present description generally describes an ocular implant or implant system, preferably an intraocular implant, including a molecular partitioning system which is administered to an eye. The intraocular implant can treat a retinal disease or condition by utilizing the molecular partitioning system to attain a controlled release of at least one therapeutic bioactive agent from the implant. With the present intraocular implant, the therapeutic bioactive agent is released into the eye for a period of time greater than about five days after the implant is placed in the eye. The implants are effective in treating or reducing at least one symptom of a retinal disease or condition, such as by increasing macular thickness, reducing retinal edema, reducing retinal vein occlusion, and/or by maintaining or improving visual acuity and color vision.

The implants described herein encompass controlled or sustained delivery of at least one therapeutic bioactive agent for the treatment of retinal diseases by direct intraocular implantation of a molecular partitioning system containing at least one therapeutic bioactive agent. The implants can further include other active agents and excipients. The at least one therapeutic bioactive agent can be released from the implants by diffusion, erosion, dissolution or osmosis and can be released from the implants over a period of about one week, ten days, fourteen days, thirty days, sixty days or up to one year. The molecular partitioning system of the implants can comprise a bioerodible polymer or polymers. The implants can be formulated as solids, semisolids or viscoelastics. Administration of the implants can be accomplished via intravitreal injection or implantation, preferably using a trocar or an applicator.

The molecular partitioning systems described herein include at least a first polymer or co-polymer and a second polymer or co-polymer. The partitioning effect is believed to occur because the first polymer or co-polymer has a mean viscosity that is at least equal to or greater than the second polymer or co-polymer. For example, a difference in mean viscosity of greater than four can be useful. Other differences in mean viscosity between the first polymer or co-polymer and the second polymer or copolymer can be at least seven or at least ten. This difference in mean viscosity causes the first and second polymer or co-polymer to partition into two different phases when formed using methods such as, but not limited to, casting and solvent evaporation. The phases themselves are further defined when at least one therapeutic bioactive agent is added to the system. The at least one therapeutic bioactive agent has an affinity for one of the two phases and partitions itself into that phase more readily than the other. The resulting system has at least two phases one being polymer rich (having less of the therapeutic bioactive agent) and a bioactive agent rich phase (having more of the therapeutic bioactive agent). In the molecular partitioning systems described herein, the at least one therapeutic bioactive agent is greater than 60% partitioned into the drug rich phase. In another example embodiment, the at least one therapeutic bioactive agent is greater than 75% partitioned into the drug rich phase.

Equally important to the controlled release proved by the molecular partitioning system, and hence the extended release profile of the implant, is the relative average molecular weight of the polymers chosen to form the implants. Molecular weight of a polymer is mathematically related to the polymers mean viscosity, and therefore, different molecular weights of the two partitioned phases or polymers are included in the implants to modulate the release profile.

Suitable polymers for use in forming the implants described herein include those which are compatible, that is biocompatible, with the eye so as to cause no substantial interference with the functioning or physiology of the eye. Such polymers preferably are at least partially and more preferably substantially completely biodegradable or bioerodible.

The polymers may be addition or condensation polymers. Generally, besides carbon and hydrogen, the polymers can include at least one oxygen. The oxygen may be present as oxy, e.g. hydroxy or ether, carbonyl, e.g. non-oxo-carbonyl, such as carboxylic acid ester, and the like.

Useful bioerodible polymers include poly(D,L-lactide-co-glycolide) (PLGA), poly(D,L-lactide) (PLA), polyesters, poly(ortho ester), poly(phosphazine), poly (phosphate ester), poly(ε-caprolactone) (PCL), natural polymers such as gelatin or collagen, or a polymeric blends.

In example embodiments, PLGA is used, where the rate of biodegradation is controlled by the ratio of glycolic acid to lactic acid. The most rapidly degraded copolymer has roughly equal amounts of glycolic acid and lactic acid. Homopolymers, or copolymers having ratios other than equal, are more resistant to degradation. The ratio of glycolic acid to lactic acid will also affect the brittleness of the resulting polymer. The percentage of polylactic acid in the PLGA copolymer can be 0-100%, preferably about 15-85%, more preferably about 35-65%. In some exemplary implants, a 50:50 PLGA copolymer is used.

Some preferred characteristics of the polymers or polymeric materials for use in the present invention may include biocompatibility, compatibility with the selected drug, ease of use of the polymer in making the drug delivery systems, a half-life in the physiological environment of at least about 6 hours, preferably greater than about one day, and water insolubility.

Release of at least one therapeutic bioactive agent from a biodegradable polymer, such as those used in the molecular partitioning systems described herein, is the consequence of several mechanisms or combinations of mechanisms. Some of these mechanisms include desorption from the implant surface, dissolution, diffusion through porous channels of the hydrated polymer and erosion. Erosion can be bulk or surface or a combination of both. In some example embodiments, therapeutic bioactive agents are released for no more than about 3-30 days after administration to the subconjunctival space. For example, an implant may comprise at least one therapeutic bioactive agent and the implant degrades at a rate effective to sustain release of a therapeutically effective amount for about one month after being placed under the conjunctiva. As another example, the implants may sustain release of a therapeutically effective amount of bioactive agent for more than thirty days, such as for about six months.

In an example embodiment, the molecular partitioning system comprises a PLA polymer having a first mean viscosity, a 50:50 PLGA polymer having a second mean viscosity and at least one therapeutic bioactive agent. A previously mentioned, the first mean viscosity is at least four times greater than the second mean viscosity. In an example embodiment, the PLA polymer has a mean viscosity between about 1.3 and about 1.7 dl/g and the PLGA polymer has a mean viscosity between about 0.32 and about 0.44 dl/g. The mean viscosities identified above may be determined in 0.1% chloroform at 25° C.

In a further example embodiment, the molecular partitioning system comprises a PLA polymer having a first molecular weight, a 50:50 PLGA polymer having a second molecular weight and at least one therapeutic bioactive agent. The first molecular weight is at least equal to or greater than the second molecular weight. For example, the first molecular weight is at least four times greater than the second molecular weight. In other examples, the first molecular weight is at least seven times, or ten times greater than the second molecular weight. The difference in molecular weight between the different polymers should allow the resulting polymer to remain stable once formed and provide the in vivo characteristics sought. In an example embodiment, the PLA polymer has a molecular weight between about 300,000 and about 100,000 Da and the PLGA polymer has a molecular weight between about 80,000 and about 10,000 Da.

The two different polymers form two different phases within the intraocular implant and the at least one therapeutic bioactive agent partitions itself into the phase containing the PLGA. The resulting phases within the molecular partitioning system are a first phase which contains the PLA polymer and is polymer rich, and the second phase contains PLGA and is rich in bioactive agent. In an example embodiment, 60% of the bioactive agent is present in the PLGA phase. In another example embodiment 75% of the bioactive agent is present in the PLGA phase.

In such an embodiment, the PLA polymer, the PLGA polymer and the at least one bioactive agent are present in the intraocular implants in predetermined ratios. In general, the PLA polymer is present from about 1% to about 80%, preferably between about 40% and about 70%; the PLGA polymer is present from about 1% to about 50%, preferably between about 10% and about 40%; and the bioactive agent is present from about 1% to about 50%, preferably between about 10% and about 30%. In one example embodiment, the implants include a ratio of PLA:PLGA:bioactive agent of about 60:20:20.

The at least one therapeutic bioactive agent used in conjunction with the intraocular implants and molecular partitioning system described herein is a tyrosine kinase inhibitor (TKI). TKIs useful according to the present description may include any compound capable of inhibiting tyrosine kinase enzymes and include compounds such as, but not limited to, axitinib, bosutinib, cediranib, dasatinib, erlotinib, gefitinib, imatinib, lapatinib, lestaurtinib, nilotinib, semaxanib, sunitinib, vandetanib and vatalanib. In one example embodiment, a TKI useful according to the present description is Compound A having the structure

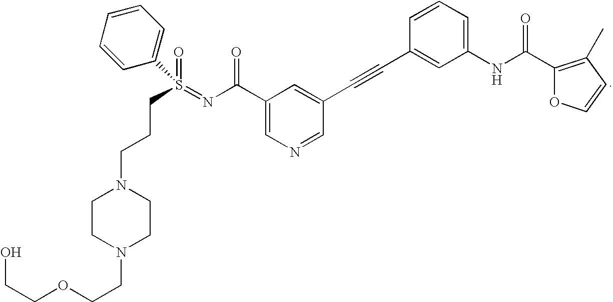

Systemic TKI induced retinal deficits, particularly when compound A is used as a TKI, are apparently be due to formation of toxic TKI metabolites. Hence a local (intraocular) administration of Compound A can prevent presentation of most if not all such toxic byproducts at a retinal target tissue. Locally delivered Compound A can have a beneficial therapeutic effect upon a retinal disease or condition. In pursuit of this therapy, intraocular implants including a molecular partitioning system and Compound A are described.

The implants described herein are developed based upon the discoveries that: (1) even though TKIs such as Compound A are eliminated from the eye extremely rapidly with half-lives of a few hours, it is theoretically feasible to deliver TKIs, for example, Compound A, to intraocular tissues at therapeutic levels over a period of, for example, one week, or for a period of time between about 2 months and about a year; (2) systemic TKI administration causes negative vision effects; (3) the negative vision effects of systemic TKI administration are probably due to metabolites generated by hepatic metabolism; (4) a method for the intraocular delivery of TKIs and their salts for the treatment of intraocular diseases is feasible; (5) a method to reduce the intraocular toxicity of locally delivered TKIs is feasible; (6) compositions of bioerodible polymeric implants and TKIs for the treatment of retinal diseases can be prepared, and; (7) compositions of bioerodible polymeric implants including a molecular partitioning system and at least one TKI with reduced local toxicity can be prepared.

Delivery of drugs or bioactive agents to the optic nerve, retina, vitreous and uveal tract is typically achieved by high systemic dosing which can cause toxicity or toxic metabolites, intra-ocular injections or other heroic measures. Penetration of systemically administered drugs into the retina is severely restricted by the blood-retinal barriers (BRB) for most compounds. As determined herein, local delivery of Compound A (in an intraocular implant with a molecular partitioning system) can prevent systemic toxicities and mitigate the BRB.

Described herein are implants which can release loads of at least one therapeutic bioactive agent over various time periods, or in other words provide controlled release of at least one TKI. These implants, which when inserted into the subconjunctival (such as a sub-tenon) space or into the vitreous of an eye provide therapeutic levels of TKI, for example Compound A, for extended periods of time (e.g., for about one week or more). The disclosed implants are effective in treating ocular conditions, such as ocular conditions associated with a retinal disease or condition, such as macula edema, macular degeneration, retinal neovascularization and retinal vein occlusion.

The implants disclosed herein can also be configured to release a TKI, for example, Compound A, with or without additional bioactive agents or drugs, to prevent or treat diseases or conditions, such as the following: maculopathies/retinal degeneration: macular degeneration, including age related macular degeneration (ARMD), such as non-exudative age related macular degeneration and exudative age related macular degeneration, choroidal neovascularization, retinopathy, including diabetic retinopathy, acute and chronic macular neuroretinopathy, central serous chorioretinopathy, and macular edema, including cystoid macular edema, and diabetic macular edema. Uveitis/retinitis/choroiditis: acute multifocal placoid pigment epitheliopathy, Behcet's disease, birdshot retinochoroidopathy, infectious (syphilis, lyme, tuberculosis, toxoplasmosis), uveitis, including intermediate uveitis (pars planitis) and anterior uveitis, multifocal choroiditis, multiple evanescent white dot syndrome (MEWDS), ocular sarcoidosis, posterior scleritis, serpignous choroiditis, subretinal fibrosis, uveitis syndrome, and Vogt-Koyanagi-Harada syndrome. Vascular diseases/exudative diseases: retinal arterial occlusive disease, central retinal vein occlusion, disseminated intravascular coagulopathy, branch retinal vein occlusion, hypertensive fundus changes, ocular ischemic syndrome, retinal arterial microaneurysms, Coat's disease, parafoveal telangiectasis, hemi-retinal vein occlusion, papillophlebitis, central retinal artery occlusion, branch retinal artery occlusion, carotid artery disease (CAD), frosted branch angitis, sickle cell retinopathy and other hemoglobinopathies, angioid streaks, familial exudative vitreoretinopathy, Eales disease. Traumatic/surgical: sympathetic ophthalmia, uveitic retinal disease, retinal detachment, trauma, laser, PDT, photocoagulation, hypoperfusion during surgery, radiation retinopathy, bone marrow transplant retinopathy. Proliferative disorders: proliferative vitreal retinopathy and epiretinal membranes, proliferative diabetic retinopathy. Infectious disorders: ocular histoplasmosis, ocular toxocariasis, presumed ocular histoplasmosis syndrome (POHS), endophthalmitis, toxoplasmosis, retinal diseases associated with HIV infection, choroidal disease associated with HIV infection, uveitic disease associated with HIV Infection, viral retinitis, acute retinal necrosis, progressive outer retinal necrosis, fungal retinal diseases, ocular syphilis, ocular tuberculosis, diffuse unilateral subacute neuroretinitis, and myiasis. Genetic disorders: retinitis pigmentosa, systemic disorders with associated retinal dystrophies, congenital stationary night blindness, cone dystrophies, Stargardt's disease and fundus flavimaculatus, Bests disease, pattern dystrophy of the retinal pigmented epithelium, X-linked retinoschisis, Sorsby's fundus dystrophy, benign concentric maculopathy, Bietti's crystalline dystrophy, pseudoxanthoma elasticum. Retinal tears/holes: retinal detachment, macular hole, giant retinal tear. Tumors: retinal disease associated with tumors, congenital hypertrophy of the RPE, posterior uveal melanoma, choroidal hemangioma, choroidal osteoma, choroidal metastasis, combined hamartoma of the retina and retinal pigmented epithelium, retinoblastoma, vasoproliferative tumors of the ocular fundus, retinal astrocytoma, intraocular lymphoid tumors. Miscellaneous: punctate inner choroidopathy, acute posterior multifocal placoid pigment epitheliopathy, myopic retinal degeneration, acute retinal pigment epithelitis and the like.

When Compound A is used in conjunction with the present implants, it is present preferably from about 1% to 90% by weight of the implants; more preferably, from about 5% to about 30% by weight of the implants. In an example embodiment, Compound A comprises about 10% by weight of the implant. In another example embodiment, Compound A comprises about 20% by weight of the implant.

The release of Compound A from an implant into the vitreous or subconjuctiva may include an initial burst of release followed by a gradual increase in the amount released, or the release may include an initial delay in release of Compound A, followed by an increase in release. When the implants are substantially completely degraded, the percent of Compound A that has been released is about one hundred. In one example embodiment, the implants described herein do not completely release, or release about 100% of Compound A, until after one week or more of being placed in an eye.

It may be desirable to provide a relatively constant rate of release of Compound A from the implants over the life of the implants. For example, it may be desirable for Compound A to be released in amounts from about 0.01 μg to about 2 μg per day for the life of the implant. However, the release rate may change to either increase or decrease depending on the formulation of the biodegradable polymer matrix. In addition, the release profile of Compound A may include one or more linear portions and/or one or more non-linear portions. Preferably, the release rate is greater than zero once the implant has begun to degrade or erode.

In some example embodiments, an implant can release about 1% of Compound A per day. In a further example embodiment, the implants may have a release rate of about 0.7% per day when measured in vitro. Thus, over a period of about 40 days, about 30% of Compound A may have been released.

The total weight of implant in a single dosage is an amount dependent on the volume of the subconjunctival space and the activity or solubility of the at least one therapeutic bioactive agent. Most often, the dose is usually about 0.1 mg to about 200 mg of implant per dose. For example, a single subconjunctival injection may contain about 1 mg, 3 mg, or about 5 mg, or about 8 mg, or about 10 mg, or about 100 mg or about 150 mg, or about 175 mg, or about 200 mg of implant, including the incorporated therapeutic bioactive agent. For non-human subjects, the dimensions and total weight of the implant may be larger or smaller, depending on the type of subject.

The dosage of therapeutic bioactive agent, for example, Compound A, in the implant is generally in the range from about 0.001 mg to about 100 mg per eye per dose, but also can vary from this depending upon the activity of the agent and its solubility.

The implants disclosed herein may have a diameter size of between about 5 μm and about 1 mm, or between about 10 μm and about 0.8 mm for administration with a needle. For needle-injected implants, the implants may have any appropriate dimensions so long as the longest dimension permits the implant to move through a needle.

The implants may be of any particulate geometry including micro and nanospheres, micro and nanoparticles, spheres, powders, rods, fragments, cubes, pills, disks, films, and the like. The upper limit for size will be determined by factors such as toleration for the implant, size limitations on insertion, desired rate of release, ease of handling, etc. Spheres may be in the range of about 0.5 μm to 4 mm in diameter, with comparable volumes for other shaped particles.

Further, the implants may have a maximum cross-section less than about 200 μm. In certain embodiments, the implants have an average or mean cross-section less than about 50 μm. In further embodiments, the cross-section ranges from about 30 μm to about 50 μm.

The size and form of the implant can also be used to control the rate of release, period of treatment, and drug concentration at the site of implantation. Larger implants will deliver a proportionately larger dose, but depending on the surface to mass ratio, may have a slower release rate. The particular size and geometry of the implant is chosen to suit the activity of the drug and the location of its target tissue.

The proportions of Compound A, polymer, and any other modifiers may be empirically determined by formulating several implant batches with varying average proportions. A USP approved method for dissolution or release test can be used to measure the rate of release (USP 23; NF 18 (1995) pp. 1790-1798). For example, using the infinite sink method, a weighed sample of implants is added to a measured volume of a solution containing 0.9% NaCl in water, where the solution volume will be such that the drug concentration is after release is less than 5% of saturation. The mixture is maintained at 37° C. and stirred slowly to maintain the implants in suspension. The appearance of the dissolved drug as a function of time may be followed by various methods known in the art, such as spectrophotometrically, HPLC, mass spectroscopy, etc. until the absorbance becomes constant or until greater than 90% of the drug has been released.

In addition to TKIs, the implants disclosed herein, the implants may also include at least one additional ophthalmically acceptable therapeutic agent or drug. For example, the implants may include one or more antihistamines, one or more antibiotics, one or more beta blockers, one or more steroids, one or more antineoplastic agents, one or more immunosuppressive agents, one or more antiviral agents, one or more antioxidant agents, and mixtures thereof. Alternatively, a single implant or injection of implants can include, in some example embodiments, two or more batches each containing a different therapeutic agent or drug in addition to the TKI.

Additional pharmacologic or therapeutic agents which may find use in the present systems further include, without limitation, those disclosed in U.S. Pat. No. 4,474,451, columns 4-6 and U.S. Pat. No. 4,327,725, columns 7-8, the entire disclosures of which are incorporated herein by reference for all that they discloses regarding pharmacologic or therapeutic agents.

Examples of antihistamines include, and are not limited to, loradatine, hydroxyzine, diphenhydramine, chlorpheniramine, brompheniramine, cyproheptadine, terfenadine, clemastine, triprolidine, carbinoxamine, diphenylpyraline, phenindamine, azatadine, tripelennamine, dexchlorpheniramine, dexbrompheniramine, methdilazine, and trimprazine doxylamine, pheniramine, pyrilamine, chiorcyclizine, thonzylamine, and derivatives thereof.

Examples of antibiotics include without limitation, cefazolin, cephradine, cefaclor, cephapirin, ceftizoxime, cefoperazone, cefotetan, cefutoxime, cefotaxime, cefadroxil, ceftazidime, cephalexin, cephalothin, cefamandole, cefoxitin, cefonicid, ceforanide, ceftriaxone, cefadroxil, cephradine, cefuroxime, ampicillin, amoxicillin, cyclacillin, ampicillin, penicillin G, penicillin V potassium, piperacillin, oxacillin, bacampicillin, cloxacillin, ticarcillin, aziocillin, carbenicillin, methicillin, nafcillin, erythromycin, tetracycline, doxycycline, minocycline, aztreonam, chloramphenicol, ciprofloxacin hydrochloride, clindamycin, metronidazole, gentamicin, lincomycin, tobramycin, vancomycin, polymyxin B sulfate, colistimethate, colistin, azithromycin, augmentin, sulfamethoxazole, trimethoprim, and derivatives thereof.

Examples of beta blockers include acebutolol, atenolol, labetalol, metoprolol, propranolol, timolol, and derivatives thereof.

Examples of steroids include corticosteroids, such as cortisone, prednisolone, flurometholone, dexamethasone, medrysone, loteprednol, fluazacort, hydrocortisone, prednisone, betamethasone, prednisone, methylprednisolone, riamcinolone hexacatonide, paramethasone acetate, diflorasone, fluocinonide, fluocinolone, triamcinolone, derivatives thereof, and mixtures thereof.

Examples of antineoplastic drugs include adriamycin, cyclophosphamide, actinomycin, bleomycin, duanorubicin, doxorubicin, epirubicin, mitomycin, methotrexate, fluorouracil, carboplatin, carmustine (BCNU), methyl-CCNU, cisplatin, etoposide, interferons, camptothecin and derivatives thereof, phenesterine, taxol and derivatives thereof, taxotere and derivatives thereof, vinblastine, vincristine, tamoxifen, etoposide, piposulfan, cyclophosphamide, and flutamide, and derivatives thereof.

Examples of immunosuppressive drugs include cyclosporine, azathioprine, tacrolimus, and derivatives thereof.

Examples of antiviral agents include interferon gamma, zidovudine, amantadine hydrochloride, ribavirin, acyclovir, valciclovir, dideoxycytidine, phosphonoformic acid, ganciclovir, and derivatives thereof.

Examples of antioxidants include ascorbate, alpha-tocopherol, mannitol, reduced glutathione, various carotenoids, cysteine, uric acid, taurine, tyrosine, superoxide dismutase, lutein, zeaxanthin, cryotpxanthin, astazanthin, lycopene, N-acetyl-cysteine, carnosine, gamma-glutamylcysteine, quercitin, lactoferrin, dihydrolipoic acid, citrate, Ginkgo Biloba extract, tea catechins, bilberry extract, vitamins E or esters of vitamin E, retinyl palmitate, and derivatives thereof.

Other therapeutic agents include squalamine, carbonic anhydrase inhibitors, alpha-2 adrenergic receptor agonists, antiparasitics, antifungals, beta-adrenergic receptor antagonists such as timolol maleate, carbonic anyhdrase inhibitors such as dorzolamide, and derivatives thereof. Combinations of any of the drugs and bioactive agents mentioned can be used according to the present description.

The amount of therapeutic bioactive agent or additional bioactive agent or drug employed in the implants, will vary widely depending on the effective dosage required and the desired rate of release from the implants. Usually the drug will be at least about 1% (w/w), more usually at least about 10% (w/w) of the implant, and usually not more than about 40% (w/w), or usually not more than about 50% (w/w) of the implants.

In addition to the therapeutic agents and drugs, the implants disclosed herein may include or may be provided in drug delivery systems that include effective amounts of buffering agents, preservatives and the like. Suitable water soluble buffering agents include, without limitation, alkali and alkaline earth carbonates, phosphates, bicarbonates, citrates, borates, acetates, succinates and the like, such as sodium phosphate, citrate, borate, acetate, bicarbonate, carbonate and the like. These agents advantageously present in amounts sufficient to maintain a pH of the system of between about 2 to about 9 and more preferably about 4 to about 8. As such the buffering agent may be as much as about 5% by weight of the total implant. Suitable water soluble preservatives include sodium bisulfite, sodium bisulfate, sodium thiosulfate, ascorbate, benzalkonium chloride, chlorobutanol, thimerosal, phenylmercuric acetate, phenylmercuric borate, phenylmercuric nitrate, parabens, methylparaben, polyvinyl alcohol, benzyl alcohol, phenylethanol and the like and mixtures thereof. These agents may be present in amounts of from about 0.001% to about 5% by weight and preferably about 0.01% to about 2% by weight. In one example embodiment, a benzylalkonium chloride preservative is provided in the implant.

In some situations mixtures of controlled release profiles within a single implant or within several different implants may be utilized employing the same or different bioactive agents. In this way, a cocktail of release profiles, giving a biphasic or triphasic release with a single administration is achieved, where the pattern of release may be greatly varied.

Additionally, release modulators such as those described in U.S. Pat. No. 5,869,079 may be included in the implants. The amount of release modulator employed will be dependent on the desired release profile, the activity of the modulator, and on the release profile of Compound A in the absence of modulator. Electrolytes such as sodium chloride and potassium chloride may also be included in the implants. Where the buffering agent or enhancer is hydrophilic, it may also act as a release accelerator. Hydrophilic additives act to increase the release rates through faster dissolution of the material surrounding the bioactive agent, which increases the surface area of the bioactive agent exposed, thereby increasing the rate of bioactive agent bioerosion. Similarly, a hydrophobic buffering agent or enhancer dissolves more slowly, slowing the exposure of bioactive agent, and thereby slowing the rate of bioactive agent bioerosion.

Further described herein are methods and process of making intraocular implants. Various techniques may be used in producing the implants described herein. Useful techniques include, but are not necessarily limited to, self-emulsification methods, super critical fluid methods, solvent evaporation methods, phase separation methods, spray drying methods, grinding methods, interfacial methods, molding methods, injection molding methods, combinations thereof and the like.

Generally, the processes for making the implants involve dissolving the appropriate polymers and bioactive agents in a solvent. Solvent selection will depend on the polymers and bioactive agents chosen. For the molecular partitioning system described herein including a bioactive agent such as Compound A, dichloromethane (DCM) is an appropriate solvent. Once the polymers and bioactive agent(s) have been dissolved, the resulting mixture is cast into a die of an appropriate shape.

Then, once cast, the solvent used to dissolve the polymers and bioactive agent(s) is evaporated at a temperature between about 20° C. and about 30° C., preferably about 25° C. The polymer can be dried at room temperature or even in a vacuum. For example, the cast polymers including bioactive agents can be dried by evaporation in a vacuum.

The dissolving and casting steps form the molecular partitioning system because dissolving the polymers and bioactive agents allows the system to naturally partition and form into its most natural configuration based on properties such as polymer viscosity and hence molecular weight, polymer hydrophobicity/hydophilicty, bioactive agent molecular weight, bioactive agent hydrophobicity/hydophilicty and the like. Conventional methods involving extrusion of dry polymer powders and dry bioactive agents will not form molecular partitioning systems as described herein because at no point are the components allowed to form the different phases as described herein. Rather, they are extruded and formed into a random orientation depending on the dry powder mix itself and not based on physical properties of the components.

Once the cast polymers are dried, they can be processed into an implant using any method known in the art to do so. In an example embodiment, the dried casted polymer can be cut into small pieces and extruded into rod shaped structures at a temperature between about 50° C. and about 120° C., preferably about 90° C. Whichever step is chosen for forming the final implants, it is preferred that the method does not substantially degrade the molecular partitioning system because it is that system that provides the controlled release of the bioactive agent(s).

The implants described herein may be inserted into the subconjunctival (i.e. sub-tenon) space or into the vitreous of an eye by a variety of methods. The method of placement may influence the therapeutic agent or drug release kinetics. A preferred means of administration of the implants is by subconjunctival injection. The location of the site of injection of the implants may influence the concentration gradients of drug surrounding the element, and thus influence the delivery rate to a given tissue of the eye. For example, an injection into the conjunctiva toward the posterior of the eye will direct drug more efficiently to the tissues of the posterior segment, while a site of injection closer to the anterior of the eye (but avoiding the cornea) may direct drug more efficiently to the anterior segment.

In an example embodiment, a method of treating a retinal disease comprises administering at least one implant containing Compound A, as disclosed herein, to a patient by subconjuctival injection. A syringe apparatus including an appropriately sized needle, for example, a 22 gauge needle, a 27 gauge needle or a 30 gauge needle, can be effectively used to inject the implant into the subconjunctival space of an eye of a human or animal. Frequent repeat injections are often not necessary due to the extended release of Compound A from the implant.

Other implants disclosed herein may be configured such that the amount of Compound A that is released from the implants within two days of subconjunctival injection is less than about 95% of the total amount of Compound A in the implants. In certain formulations, 95% of Compound A is not released until after about one week of injection. In certain implant formulations, about 50% of compound A is released within about one day of placement in the eye, and about 2% is released for about 1 month after being placed in the eye. In other example embodiments, about 50% of Compound A is released within about one day of subconjunctival administration, and about 1% is released for about 2 months after such administration.

The implants may further be administered to patients in conjunction with or in a composition with an ophthalmically acceptable liquid composition, suspension, emulsion, and the like, and administered by injection or implantation into the subconjunctival space of the eye. The implants described herein can further be formulated into a composition with a high viscosity, polymeric gel to reduce dispersion of one or more implants upon intraocular injection. Preferably, the gel has a high shear characteristic, meaning that the gel can be injected into an intraocular site through a 25-30 gauge needle, and more preferably through a 27-30 gauge needle. A suitable gel for this purpose can be a hydrogel or a colloidal gel formed as a dispersion in water or other aqueous medium. Examples of suitable gels include synthetic polymers such as polyhydroxy ethyl methacrylate, and chemically or physically crosslinked polyvinyl alcohol, polyacrylamide, poly(N-vinyl pyrolidone), polyethylene oxide, and hydrolysed polyacrylonitrile. Examples of suitable hydrogels which are organic polymers include covalent or jonically crosslinked polysaccharide-based hydrogels such as the polyvalent metal salts of alginate, pectin, carboxymethyl cellulose, heparin, hyaluronate and hydrogels from chitin, chitosan, pullulan, gellan, xanthan and hydroxypropylmethylcellulose. Commercially available dermal fillers (such as HYLAFORM® (Biomatrix, Inc., Ridgefiled, N.J.), RESTYLANE® (HA North American Sales, Scottsdale, Ariz.), Sculptura and RADIESSE® (BioForm Medical, Inc., San Mateo, Calif.)) can be used as the high viscosity gel.

Hyaluronic acid (HA) is a polysaccharide made by various body tissues and can also be used a high viscosity, polymeric gel to reduce dispersion of one or more implants upon intraocular injection. U.S. Pat. No. 5,166,331 discusses purification of different fractions of HA for use as a substitute for intraocular fluids and as a topical ophthalmic drug carrier. Other U.S. patent applications which discuss ocular uses of HA include Ser. Nos. 11/859,627; 11/952,927; 10/966,764; 11/741,366; and 11/039,192 The pharmaceutical compositions described herein preferably comprise a high viscosity HA with an average molecular weight between about 1 and 4 million Daltons, and more preferably with an average molecular weight between about 2 and 3 million Daltons, and most preferably with an average molecular weight of about (±10%) 2 million Daltons.

Dry uncross-linked HA material comprises fibers or powder of commercially available HA, for example, fibers or powder of sodium hyaluronate (NaHA). The HA may be bacterial-sourced NaHA, animal derived NaHA or a combination thereof. In some example embodiments, the dry HA material is a combination of raw materials including HA and at least one other polysaccharide, for example, glycosaminoglycan (GAG).

In some example embodiments, the HA compositions comprise or consist of high molecular weight HA. That is, nearly 100% of the HA material in the compositions is a high molecular weight HA. High molecular weight HA means HA with a molecular weight of at least about 1.0 million Daltons (mw≧10⁶ Da) to about 4.0 million Da (mw≦4×10⁶ Da). For example, the high molecular weight HA in the present compositions may have a molecular weight of about 2.0 million Da (mw 2×10⁶ Da). In another example embodiment, the high molecular weight HA may have a molecular weight of about 2.8 million Da (mw 2.8×10⁶ Da).

In another example embodiment, HA compositions are produced using dry, raw HA material, for example, NaHA, having a desired high/low molecular weight ratio. First, the dry, raw HA material is cleaned and purified. These steps generally involve hydrating the dry HA fibers or powder in the desired high/low molecular weight ratio, for example, using pure water, and filtering the material to remove large foreign matters and/or other impurities. The filtered, hydrated material is then dried and purified. The high and low molecular weight NaHA may be cleaned and purified separately, or may be mixed together, for example, in the desired ratio, just prior to cross-linking.

At this stage in the process, the pure, dried NaHA fibers are hydrated in an alkaline solution to produce an uncross-linked NaHA alkaline gel. Any suitable alkaline solution may be used to hydrate the NaHA in this step, for example, but not limited to an aqueous solution containing NaOH. The resulting alkaline gel will have a pH above 7.5, for example, a pH above 8, for example, a pH above 9, for example, a pH above 10, for example, a pH above 12, for example, a pH above 13.

In one example embodiment, the next step in the manufacturing process comprises the step of cross-linking the hydrated, alkaline NaHA gel with a suitable cross-linking agent, for example, butanediol diglycidyl ether (BDDE).

The step of HA cross-linking may be carried out using means known to those of skill in the art. Those skilled in the art appreciate how to optimize the conditions of cross-linking according to the nature of the HA, and how to carry out the cross-linking to an optimized degree. In some example embodiments, the degree of cross-linking is at least about 2% to about 20%, for example, is about 4% to about 12%, wherein the degree of cross-linking is defined as the percent weight ratio of the cross-linking agent to HA-monomeric units in the HA composition.

The hydrated cross-linked, HA gel may be neutralized by adding an aqueous solution containing HCl. The gel is then swelled in a phosphate buffered saline solution for a sufficient time and at a low temperature.

In certain example embodiments, the resulting swollen HA gel is a cohesive gel having substantially no visible distinct particles, for example, substantially no visibly distinct particles when viewed with the naked eye. In some embodiments, the gel has substantially no visibly distinct particles under a magnification of less than 35×.

The HA gel is now purified by conventional means for example, dialysis or alcohol precipitation, to recover the cross-linked material, to stabilize the pH of the material and remove any unreacted cross-linking agent. Additional water or slightly alkaline aqueous solution can be added to bring the concentration of the NaHA in the composition to a desired concentration. In some embodiments, the concentration of NaHA in the composition is in a range between about 10 mg/ml to about 30 mg/ml.

The implants dissolved within a HA composition and injected into the eye can have controlled release of the at least one therapeutic bioactive agent provided by the molecular partitioning system and further by the HA itself. In some example embodiments, the HA can delay release of the bioactive agent by 3 months, and therefore, controlled release of the bioactive agent can be delayed once implanted. In other example embodiments, the HA can help achieve further fine tuning to the controlled release provide by the molecular partitioning system.

### EXAMPLE 1

### A. Implant Preparation

Compound A and various amounts of RESOMER® RG502 a 50:50 poly(D,L-lactide-co-glycolide) (PLGA) polymer with an inherent viscosity of about 0.16-0.24 dl/g, RESOMER® RG503H a 50:50 PLGA polymer with an inherent viscosity of about 0.32-0.44 dl/g, RESOMER® R207 a poly(D,L-lactide) (PLA) polymer with an inherent viscosity of about 1.3-1.7 dl/g, RESOMER® R203 a PLA polymer with an inherent viscosity of about 0.25-0.35 dl/g and poly(ε-caprolactone) (PCL) were formed into polymeric implants including a molecular partitioning system according to Tables 1 and 2.

TABLE 1
Composition (%) of the implant formulations for the in vitro tests

| Lot number | Compound A | R207 | RG503H | RG502 | PCL | R203 |
|---|---|---|---|---|---|---|
| Implant 1 | 20 | 40 | 40 | 0 | 0 | 0 |
| Implant 2 | 20 | 40 | 0 | 40 | 0 | 0 |
| Implant 3 | 10 | 45 | 0 | 45 | 0 | 0 |
| Implant 4 | 20 | 0 | 0 | 0 | 80 | 0 |
| Implant 5 | 20 | 60 | 20 | 0 | 0 | 0 |
| Implant 6 | 20 | 0 | 0 | 0 | 0 | 80 |

TABLE 2
Composition (%) of the implant formulations for the in vivo tests

| Lot number | Compound A | R207 | RG503H | PCL |
|---|---|---|---|---|
| Implant 7 | 0 | 50 | 50 | 0 |
| Implant 8 | 20 | 40 | 40 | 0 |
| Implant 9 | 0 | 75 | 25 | 0 |
| Implant 10 | 20 | 60 | 20 | 0 |
| Implant 11 | 0 | 0 | 0 | 100 |
| Implant 12 | 20 | 0 | 0 | 80 |

The polymers were accurately weighed according to the formulas given in Table 1 and 2, mixed and dissolved in 4 mL dichloromethane (DCM). The resulting solutions were cast into TEFLON® (Du Pont, Willmington, Del.) dishes and dried in a fume hood for 20 hours and then in a vacuum oven for additional 3 hours. The dried membranes were cut into small pieces and extruded into filaments using a piston extruder A nozzle with a diameter of 440 μm was used. The extrusion temperatures were 90° C. for the formulations containing PLA and PLGA and 75° C. for those containing PCL. The filaments were cut into 7 mm long implants for both in vitro release tests and in vivo evaluation. The implants for in vivo evaluation were loaded into applicators and packed individually in aluminum foil bags and provided to the animal test group. Every caution was taken in the preparation and packaging processes to avoid any potential contamination. No further sterilization was performed.

### EXAMPLE 2

### In Vitro Release of Compound A

In vitro release analysis was carried out in an incubator at 37° C. shaking at 120 rpm. The release medium was 0.02% Polysorbate 80 containing 10 mM phosphate buffered saline, pH 7.4. The medium and implants were placed in 20 mL scintillation vials. At given time points, the medium containing released Compound A was collected and replaced with fresh medium. The concentration of Compound A in the release medium was analyzed using high performance liquid chromatography (HPLC).

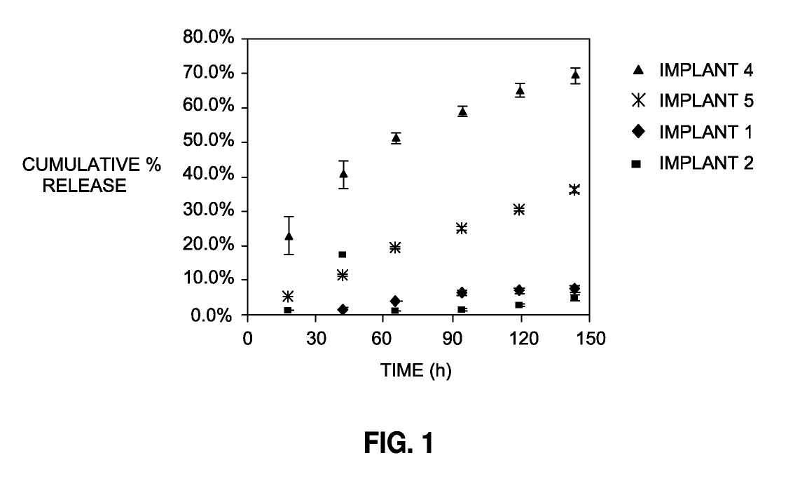

Release profiles of Implants 1, 2, 5 and 6 prepared in Example 1 were evaluated in vitro and results are graphically illustrated in FIG. 1. All the 4 implants contained 20% Compound A. The releases within the first 6 days from Implants 1 and 2, both containing 40% PLGA and 40% PLA followed close to zero-order kinetics and the average cumulative releases were 7.5% and 5.0%, respectively. The release from Implant 5 containing 60% PLA and 20% PLGA followed similar kinetics, but the cumulative release was much higher with a total of 36.4% initially loaded Compound A released during the same period of time. The release rate of Implant 4, containing 80% PCL, was the highest at the beginning, approximately four times as high as the average rate of the Implant 5 in the first day, but decreased rapidly to a comparable level after four days. Implant 3 and 4 had negligible release in 6 days (data were not shown).

Based on these results, the following three formulations were selected for in vivo evaluation to provide low, medium, and high release rates:
Slow release formulation: Implant 1, 40% R207, 40% RG503H, and 20% Compound A;
Medium release formulation: Implant 5, 60% R207, 20% RG503H, and 20% Compound A;
Fast release formulation: Implant 6, 80% PCL and 20% Compound A.

Implants 1, 5 and 6 were selected for evaluation in a rabbit model of VEGF-induced retinal vasculopathy. The slow (Implant 1) and medium (Implant 5) release implants were used for pharmacodynamic and safety evaluations and were retrieved after 5 days in rabbit eyes for determination of residual compound and physicochemical characterization. The residual amounts of Compound A in the implants were determined and the results are shown in Table 3.

The average cumulative releases were 18.7±1.1% (n=4) for the slow release implants and 45.2%±1.7% (n=4) for the medium release implants. Assuming zero-order release kinetics, the average release rates in the rabbit eyes were 8.0 μg/day and 21.8 μg/day, respectively.

TABLE 3
Cumulative release of Compound A from the implants
in rabbit eyes for 5 days

| | Initial API | Residual API | | Amount |
|---|---|---|---|---|
| Lot number | content (μg) | content (μg) | % Released | released (μg) |
| Implant 13 | 213.52 | 173.8 | 18.6% | 39.7 |
| Implant 14 | 230.79 | 188.7 | 18.2% | 42.1 |
| Implant 15 | 213.52 | 175.6 | 17.7% | 37.9 |
| Implant 16 | 199.39 | 159.0 | 20.3% | 40.4 |
| | | Mean | 18.7% | 40.0 |
| | | Stdev | 1.1% | 1.7 |
| Implant 17 | 240.12 | 127.4 | 47.0% | 112.7 |
| Implant 18 | 229.68 | 130.9 | 43.0% | 98.8 |
| Implant 19 | 269.7 | 145.6 | 46.0% | 124.1 |
| Implant 20 | 224.46 | 123.9 | 44.8% | 100.5 |
| | | Mean | 45.2% | 109.0 |
| | | Stdev | 1.7% | 11.8 |

Both in vitro and in vivo release results indicated that the implants containing 40% PLA and 40% PLGA released slower than the ones containing 60% PLA and 20% PLGA. Generally speaking, lower glass transition temperatures (T_{g}) of polymers leads to faster release. These results were counter-intuitive as a result of the fact that the T_{g} of the PLGA used was lower than the T_{g} of PLA used.

### EXAMPLE 3

### Surface Morphology of the Implants

The surface morphology of the implants was examined using scanning electron microscopy (SEM). A Zeiss EVO 40 microscope was used. The samples were coated with a thin layer of gold using a K550X Sputter Coater (Emitech Ltd., Kent, UK). The images were acquired using a secondary electron detector.

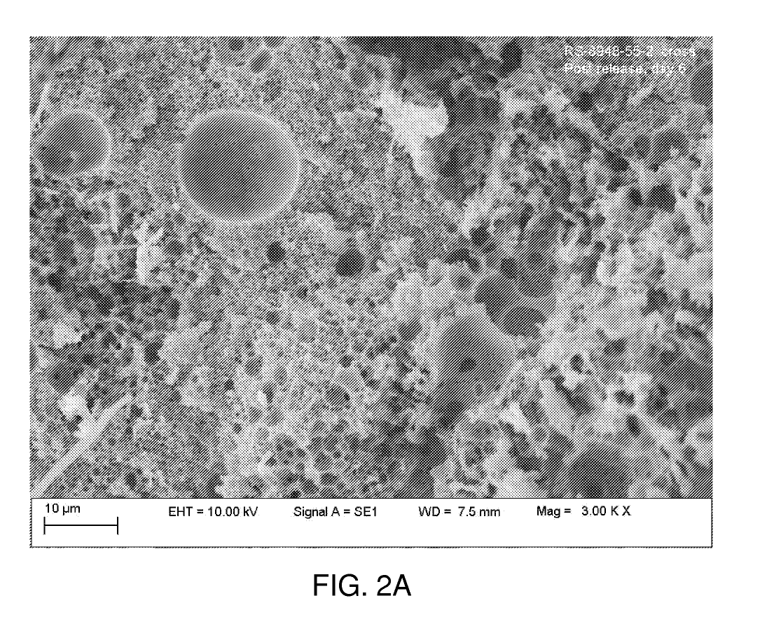

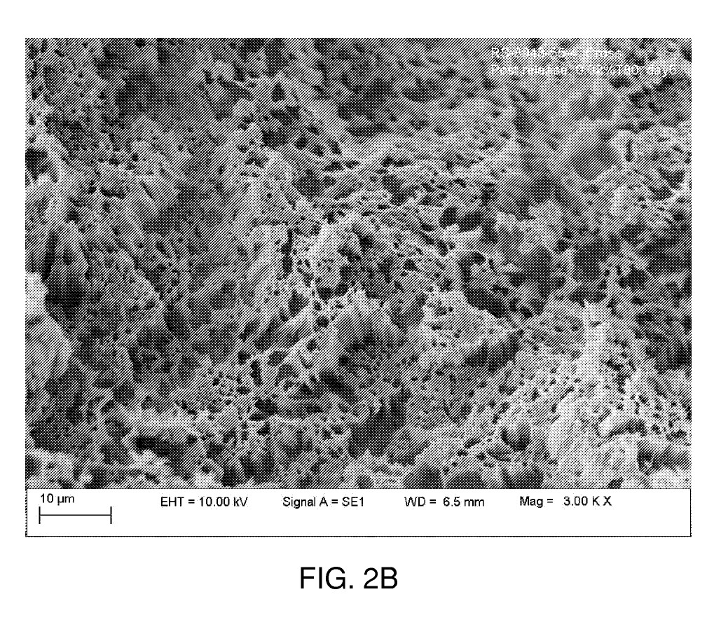

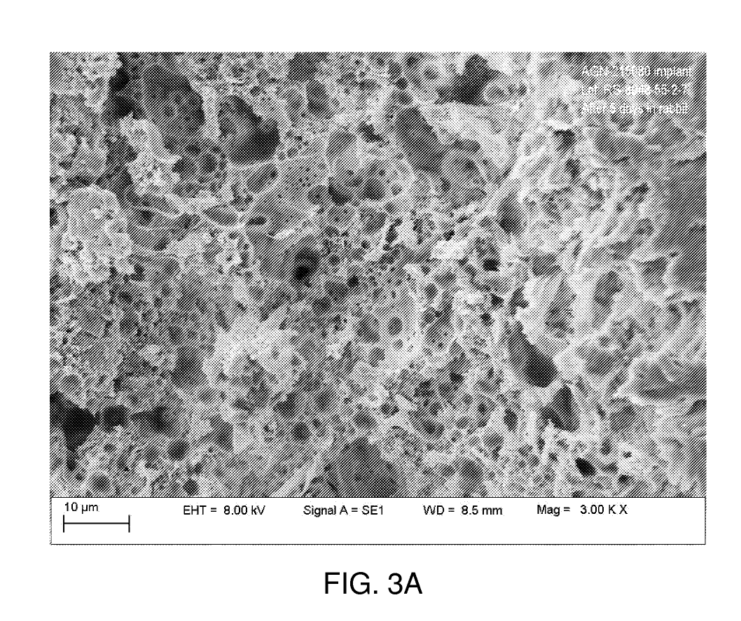

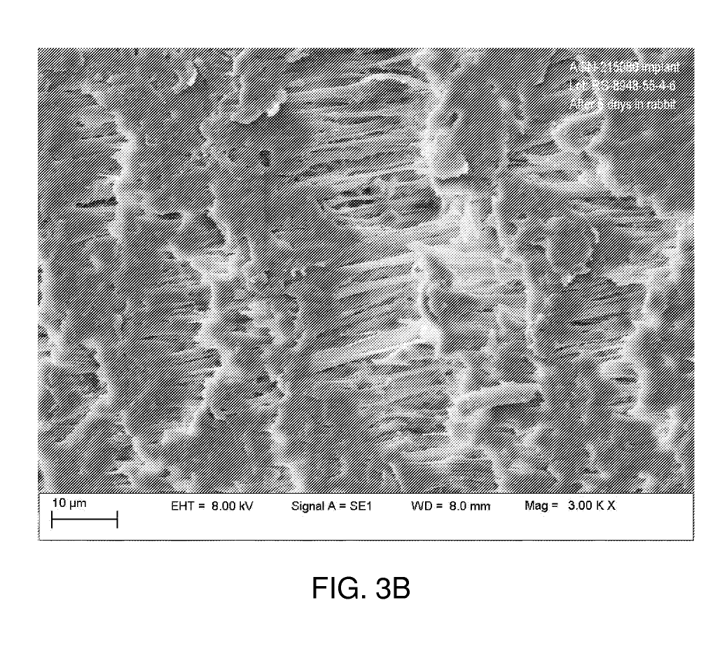

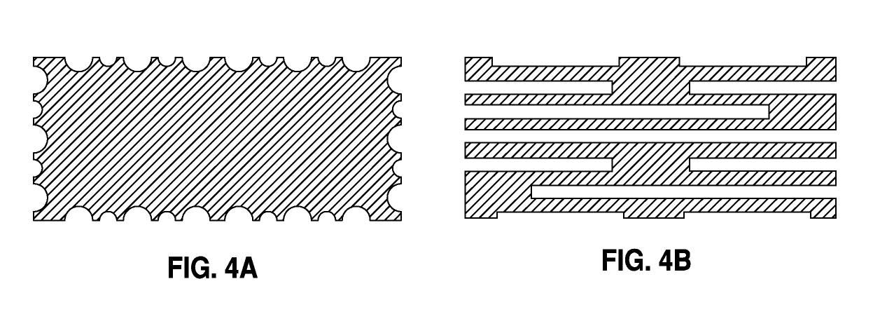

To understand the release mechanism of the molecular partitioning system, SEM was used to examine the surface morphology of the implants before and after release. Before release, no pore was found on the surface of the implants and very few pores were found on cross-section of the implants, which might have originated from air trapped during the extrusion process. SEM images of the implants after in vitro release and in vivo release are shown in FIGS. 2 and 3, respectively. The images indicated that large numbers of pores formed during the releases and most importantly the shapes of the pores in the two types of implants were different. The pores in the slow release implants were mostly spherical while those in the medium release implants were tubular. Diagrammatic representations of these pores are shown in FIGS. 4A and 4B. The tubular pores in FIG. 4B led to a more dramatic increase in the surface area of the implants than the spherical pores in FIG. 4A. The difference in surface areas at least partially caused the difference in release rates.

### EXAMPLE 4

### Degradation of the Polymers

Degradation of polymers of the implants in vitro and in vivo, within rabbit eyes, was examined using gel permeation chromatography (GPC). The instrument components and operation conditions were:
Alliance 2695 Liquid Chromatography system;
Waters 2414 Refractive Index Detector;
Columns: STYRAGEL® ((Waters Technologies Corp, Wilmington, Del.) HR4E and HR5 (7.8×300 mm) in tandem;
Mobile phase: tetrahydrofuran (THF);
Temperature for the columns and detector: 35° C.;
Flow rate: 1 mL/min.

The columns were calibrated using polystyrene standards. The polymer raw materials, the cast membranes of the formulations, and the implant samples before and after release or implantation were dissolved in THF and analyzed.

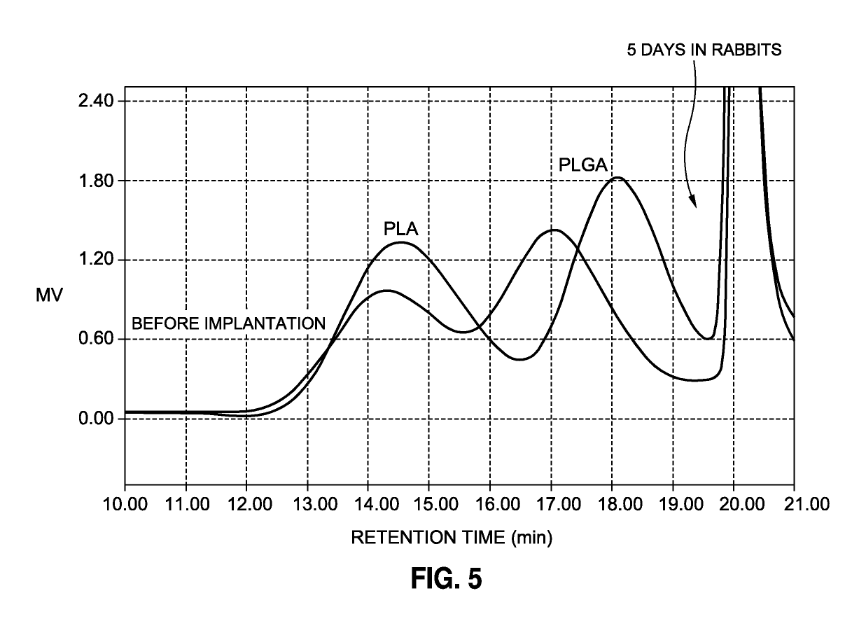

Polymer degradation was examined using GPC. The GPC chromatograms shown in FIG. 5 indicated that both PLA and PLGA degraded after 5 days implantation in rabbit eyes. However, the degradation was more significant for PLGA than for PLA. During the 5 days in the rabbit eyes, the relative molecular weight decreased more than 60% for PLGA compared to less than 20% for PLA. Similar results were obtained for the implants after 6 days in vitro release tests.

### EXAMPLE 5

### In Vivo Release Rate

The in vivo release rate of Compound A in rabbit eyes was estimated by determining the residual content of Compound A in retrieved implants after being implanted for 5 days. The retrieved implants were dried under vacuum for 20 hours. Each of the implants was dissolved in 4 mL DCM in a 20 mL scintillation vial. The solutions were dried in a fume hood and 10 mL of 50% acetonitrile in water was added to each vial to extract Compound A. The concentration of Compound A was analyzed using HPLC.

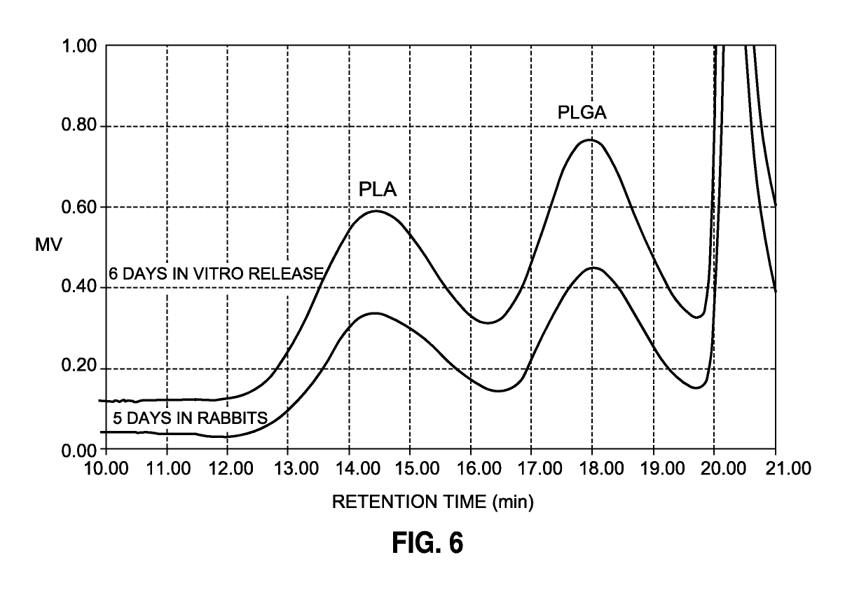

The degradation rate in vivo was found very close to that in vitro. A comparison of the GPC chromatograms of the implants after 5 days in rabbit eyes and after 6 days in vitro release is shown in FIG. 6. The results suggested that hydrolysis was the predominant degradation mechanism of PLA and PLGA in rabbit eyes, and the degradation rate in rabbit eyes could be predicted by in vitro degradation results.

### EXAMPLE 6

### Treatment of Neovascularization

A 68 year old woman complains of blurry vision in her left eye and is seen by her general ophthalmologist. She has visual acuity of CF 3 ft left eye with an ischemic central retinal vein occlusion with numerous cotton wool spots apparent in the posterior pole. The patient is watched closely and develops macula neovascularization 3 months following the vein occlusion. The intraocular pressure (IOP) increases to 42 mmHg and the angle can show fine new vessels coursing through the retina, trebecular meshwork with anterior synechiae noted temporally. The patient can receive a subTenon's or intravitreal injection of a slow release implant of Example 2. After 2 weeks, the IOP can be 26 mmHg both the iris and retinal neovascularization neovascularization improved.

### EXAMPLE 7

### Treatment of Macular Degeneration

A 76 year old man has age-related macular degeneration and cataracts in both eyes. The patient can also have a history of cardiovascular disease and an inferior wall myocardial infarction 6 months previous. The patient can complain of blurry vision and metamorphopsia in the right eye and examination can reveal visual acuity of 20/400 right eye, 20/32 left eye. Retinal examination can show subfoveal choroidal neovascularization (CNV) (right eye wet AMD) approximately 1 disc area in size with surrounding hemorrhage and edema in the right eye. The fellow left eye can show high-risk features for developing wet AMD such as soft, amorphic appearing drusen that included the fovea but no signs of choroidal neovascularization and can be confirmed by fluorescein angiography (left eye dry AMD).

In both eyes the patient can receive an intravitreal injection of a slow release implant of Example 2. The patient can receive intravitreal left eye injections of the slow release implant of Example 2 every 6 months and at the end of a 7-year follow up period the patient can have maintained vision in the both eyes of at least 20/32.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

Specific example embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Example embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

We claim:

1. An intraocular implant for treating an ocular condition, the implant comprising:
a molecular partitioning system comprising a poly(D,L-lactide) phase having a first inherent viscosity; a poly(D,L-lactide-co-glycolide) phase having a second inherent viscosity; and at least one therapeutic bioactive agent;
wherein said first mean viscosity is at least about four times greater than said second mean viscosity, and wherein said molecular partitioning system provides controlled release of said at least one therapeutic bioactive agent from said intraocular implant.

2. The intraocular implant according to claim 1 wherein said poly(D,L-lactide) phase has a first molecular weight and said poly(D,L-lactide-co-glycolide) phase has a second molecular weight wherein said first molecular weight is at least four times greater than said second molecular weight.

3. The intraocular implant according to claim 1 wherein said poly(D,L-lactide) phase, said poly(D,L-lactide-co-glycolide) phase, and said at least one therapeutic bioactive agent are present at a ratio of about 60:20:20.

4. The intraocular implant according to claim 1 wherein said at least one therapeutic bioactive agent is a tyrosine kinase inhibitor having the structure

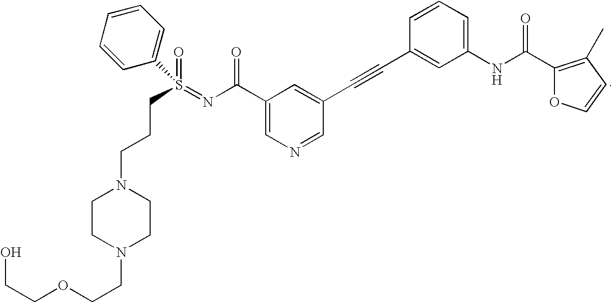

5. The intraocular implant according to claim 1 wherein said at least one therapeutic bioactive agent is greater than about 60% partitioned into said poly(D,L-lactide-co-glycolide) phase.

6. The intraocular implant according to claim 1 wherein said at least one therapeutic bioactive agent is greater than about 75% partitioned into said poly(D,L-lactide-co-glycolide) phase.

7. A process for making an intraocular implant having a molecular partition system comprising:
dissolving a poly(D,L-lactide) polymer having a first mean viscosity; a poly(D,L-lactide-co-glycolide) polymer having a second mean viscosity; and at least one therapeutic bioactive agent in a solvent thereby forming a mixture;
casting said mixture;
evaporating said solvent thereby forming a polymeric film comprising said molecular partitioning system, said molecular partitioning system comprising a poly(D,L-lactide) phase having said first mean viscosity and a poly(D,L-lactide-co-glycolide) phase having said second mean viscosity; and
extruding said polymer film thereby making said intraocular implant,
wherein said first mean viscosity is at least about four times greater than said second mean viscosity and said molecular partitioning system provides controlled release of said at least one therapeutic bioactive agent from said intraocular implant.

8. The method according to claim 7 wherein said poly(D,L-lactide) polymer, said poly(D,L-lactide-co-glycolide) polymer, and said at least one therapeutic bioactive agent are present at a ratio of about 60:20:20.

9. The method according to claim 7 wherein said at least one therapeutic bioactive agent is a tyrosine kinase inhibitor having the structure

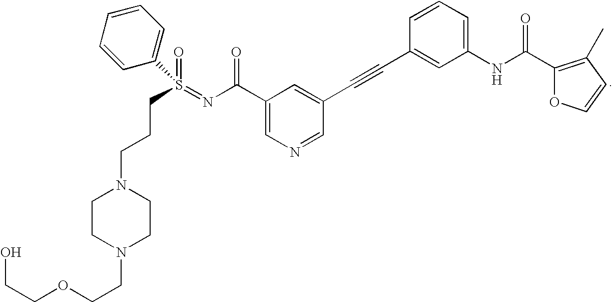

10. The method according to claim 7 wherein said at least one therapeutic bioactive agent is greater than about 60% partitioned into said poly(D,L-lactide-co-glycolide) phase.

11. The method according to claim 7 wherein said at least one therapeutic bioactive agent is greater than about 75% partitioned into said poly(D,L-lactide-co-glycolide) phase.

12. The method according to claim 7 wherein said extruding step is performed at a temperature of about 90° C.

13. A method of treating an ocular condition comprising the steps of:
(a) selecting a patient with an ocular condition in need of treatment;
(b) providing an intraocular implant comprising a molecular partitioning system comprising a poly(D,L-lactide) phase having a first mean viscosity; a poly(D,L-lactide-co-glycolide) phase having a second mean viscosity; and at least one therapeutic bioactive agent, wherein said first mean viscosity is at least about four times greater than said second mean viscosity, and wherein said molecular partitioning system provides controlled release of said at least one therapeutic bioactive agent from said intraocular implant;
(c) inserting said intraocular implant into a region of an eye; and
(d) treating said ocular condition.

14. The method of treating an ocular condition according to claim 13 wherein said poly(D,L-lactide) phase, said poly(D,L-lactide-co-glycolide) phase, and said at least one therapeutic bioactive agent are present at a ratio of about 60:20:20.

15. The method of treating an ocular condition according to claim 13 wherein said at least one therapeutic bioactive agent is a tyrosine kinase inhibitor having the structure

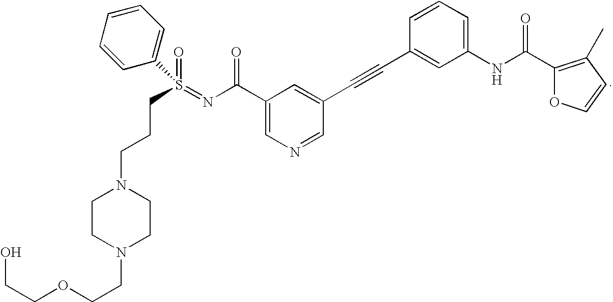

16. The method of treating an ocular condition according to claim 13 wherein said at least one therapeutic bioactive agent is greater than about 60% partitioned into said poly(D,L-lactide-co-glycolide) phase.

17. The method of treating an ocular condition according to claim 13 wherein said at least one therapeutic bioactive agent is greater than about 75% partitioned into said poly(D,L-lactide-co-glycolide) phase.

18. The method of treating an ocular condition according to claim 13 wherein said ocular implant is rod shaped.

19. A process of making an intraocular implant having a molecular partitioning system comprising:
dissolving a poly(D,L-lactide) polymer having a mean viscosity between about 1.3 and about 1.7 dl/g ; a poly(D,L-lactide-co-glycolide) polymer having a mean viscosity between about 0.32 and about 0.44 dl/g; and at least one bioactive agent in dichloromethane thereby forming a mixture;
casting said mixture;
evaporating said dichloromethane thereby forming a polymer film comprising said molecular partitioning system having a poly(D,L-lactide) phase and a poly(D,L-lactide-co-glycolide) phase; and
extruding said polymeric film into rod shaped structures at a temperature of about 90° C. thereby making said intraocular implant,
wherein said molecular partitioning system provides controlled release of said at least one bioactive agent from said intraocular implant.

20. The method of making an intraocular implant according to claim 19 wherein said poly(D,L-lactide) polymer, said poly(D,L-lactide-co-glycolide) polymer, and said at least one bioactive agent are present at a ratio of about 60:20:20.
